(19) United States
(12) Patent Application Publication
(10) Pub. No.: US 2025/0308716 A1
(43) Pub. Date: Oct. 2, 2025

(54) SUPPORT PLATE FOR TUBES IN A REACTOR VESSEL

(71) Applicant: L'Air Liquide, Société Anonyme pour I'Etude et I'Exploitation des Procédés Georges Claude, Paris (FR)

(72) Inventors: Tobias OELMANN, Bad Vilbel (DE); Katja Bartels, Frankfurt am Main (DE); Brigitte Mueller, Frankfurt am Main (DE); Veronika Gronemann, Karben (DE); Michael Strozyk, Offenbach (DE)

(21) Appl. No.: 19/089,079

(22) Filed: Mar. 25, 2025

(30) Foreign Application Priority Data
Mar. 28, 2024 (EP) 24167622

(51) Int. Cl. G21C 13/02; C07C 29/152

(52) U.S. Cl. CPC G21C 13/02; C07C 29/152

(57) ABSTRACT

A device (1.1, 1.2) with a reactor vessel (2), a tube bundle (3) of multiple tubes (4), and at least one support plate (5), wherein the tube bundle (3) is disposed in the reactor vessel (2), wherein the support plate (5) is disposed in the reactor vessel (2) transversely to a longitudinal axis (6) of the reactor vessel (2), wherein each tube (4) of the tube bundle (3) is routed through a respective tube opening (7) of the support plate (5), wherein the support plate (5) supports the tubes (4) of the tube bundle (3) in the tube openings (7) transversely to the longitudinal direction of the tubes (4), wherein the support plate (5) has fluid-exchange cutouts (8) between the tube openings (7).

### CROSS REFERENCE TO RELATED APPLICATIONS

This application claims the benefit of priority under 35 U.S.C. § 119 (a) and (b) to European Patent Application No. EP24167622, filed Mar. 28, 2024, the entire contents of which are incorporated herein by reference.

### BACKGROUND

The invention relates to an assembly for methanol synthesis and to a device that can be part of such an assembly. The invention also relates to the use of the device and to a process for methanol synthesis using the device.

Processes for the industrial preparation of methanol by heterogeneously catalysed conversion of synthesis gas in suitable synthesis reactors are known. Synthesis gases may be different gas mixtures which include hydrogen and carbon dioxide. The reactor is usually in the form of an upright fixed-tube heat exchanger.

Two-stage processes for preparing methanol are also known. In these processes, synthesis gas is fed to a water-cooled reactor and then to a gas-cooled reactor. A copper-based solid-bed catalyst is used to convert the synthesis gas to methanol.

In the case of a water-cooled reactor, the catalyst is inside the tubes, surrounded by water or steam on the shell side. The tubes are mechanically fixed via tube plates and supporting metal sheets in the reactor.

The cooling in the water-cooled reactor is performed via heat being released into the water, whereupon steam can be produced. The steam-water mixture rises on the tubes. The supporting metal sheets must ensure the fixing of the tubes. Supporting metal sheets inserted in offset fashion in the reactor vessel, such that the coolant flows in a meandering course in the reactor vessel around the supporting metal sheets, are known.

Depending on the selected geometries of the heat-transferring components and of the mechanical elements of the coolant side, a pressure drop is generated on the coolant side in the steam. It is primarily long reactor tubes which result in a higher pressure drop.

Frequently, the external dimensions of the reactor are restricted for transport, and this leads to long, slender reactors. However, the consequence of this is an elevated pressure drop on the coolant side. The long shape of the reactor means further support plates are required to avoid the tubes sagging when they are being transported, and also avoid them bowing, bending and swaying when in operation.

### SUMMARY

An object of the present invention, proceeding from the described prior art, is to reduce the hindering effect of the support plates and at the same time prevent the tubes from bending, bowing or swaying.

This object is achieved by the independent claims. Further advantageous configurations are specified in the dependent claims. The features set out in the claims and in the description may be combined with one another in any technologically appropriate way.

The invention sets out a device which comprises a reactor vessel, a tube bundle of multiple tubes, and at least one support plate. The tube bundle is located in the reactor vessel. The support plate is disposed in the reactor vessel transversely to a longitudinal axis of the reactor vessel and each tube of the tube bundle is routed through a respective tube opening of the support plate. The support plate supports the tubes of the tube bundle in the tube openings transversely to the longitudinal direction of the tubes. Furthermore, the support plate has fluid-exchange cutouts between the tube openings.

The device is preferably in the form of a reactor. The device may be designed to carry out a chemical reaction. The chemical reaction may be an exothermic or endothermic reaction. The device is particularly suitable for methanol synthesis.

However, the advantages described here can also be achieved for numerous other chemical reactions. The advantages can even be achieved if the device is not used as a reactor and no chemical reaction proceeds in the device. The device may in general be in the form of a heat exchanger.

The device comprises a reactor vessel and a tube bundle of multiple tubes in the reactor vessel. A first medium can flow through the tubes. Outside the tubes there may be a second medium in the reactor vessel. The second medium can flow through intermediate spaces between the tubes. Between the first medium and the second medium, an exchange of heat can take place. The first medium and the second medium can flow in opposite directions. In this case, the device is operated in a counter-current configuration. The first medium and the second medium can, however, also flow in the same direction. In the case of methanol synthesis, the first medium may contain for example the reaction reactants and the second medium may be a cooling medium, or vice versa.

The reactor vessel is preferably an elongate hollow body. The reactor vessel may be a cylindrical metal vessel suitable for encompassing the tube bundle. The reactor vessel may have ports and connections for fluidically connecting the tube bundle. Furthermore, the reactor vessel may have ports and connections so that a cooling medium or other medium can be introduced into and discharged from the reaction vessel into or from a shell space outside and between the tubes. The shell space may be fluidically connected.

The tube bundle comprises multiple tubes. The tube bundle preferably comprises at least 6 tubes. The tube bundle preferably comprises at least 100 tubes. The tube bundle particularly preferably comprises even at least 1000 tubes.

The tubes may be elongate cylindrical hollow bodies with a uniform wall thickness. The wall thickness of the tubes is preferably at most 5 mm, more preferably at most 3 mm and particularly preferably at most 0.1 mm. The tubes may be made in particular of a metallic material. The material preferably conducts heat.

Some of the tubes or all of the tubes preferably contain a catalyst. In the case of methanol synthesis, it is thus possible for example to conduct the reaction reactants through the tubes and react them with the catalyst in the tubes so as to form methanol.

The reaction reactants may be provided in particular in the form of a synthesis gas. Preferred reaction reactants are hydrogen, carbon monoxide and carbon dioxide. A mixture of hydrogen and carbon monoxide is preferred. A mixture of hydrogen and carbon dioxide is particularly preferred. Furthermore, the synthesis gas may contain inert gases. In particular, the synthesis gas may contain methane as inert gas. The synthesis gas preferably contains nitrogen as inert gas. The synthesis gas can be conducted into the tubes on an inlet side and react with the catalyst in the tube. In the case of an exothermic reaction, heat generated during the reaction can be released via the tube shell. A coolant can discharge the heat, preferably by convection, from the shell side. The reaction product formed in the tube can, together with the remaining synthesis gas, be discharged on the outlet side.

As an alternative, a synthesis gas with the reaction reactants can also be conducted through the reaction vessel outside the tubes. In particular in this case, a catalyst bed may be provided on the shell side in the shell space. The synthesis gas can then be passed through the catalyst bed. A coolant can be conducted into the tubes on an inlet side. The heat generated by the reaction can be transferred to the coolant in the tubes via the shell side of the tubes. The heated coolant can be discharged on an outlet side of the tubes.

The device also comprises the support plate, which supports the tubes in the reactor vessel. The support plate may be in the form of a metal sheet. The support plate preferably has a thickness of at least 7 mm. The thickness of the support plate is preferably at most half of a diameter of the tubes. The optimum thickness of the support plate can also be ascertained via calculations. If not all the tubes have the same diameter, the thickness of the support plate is preferably at most half of the largest diameter of the tubes of the tube bundle.

The support plate is disposed in the reactor vessel transversely to a longitudinal axis of the reactor vessel. The longitudinal axis of the reactor vessel is preferably aligned along the elongate direction of the hollow body. The reactor vessel and/or the tube bundle is preferably vertically aligned. The “and” version is preferred.

The support plate has tube openings for passage of the tubes of the tube bundle. Each tube of the tube bundle is routed through a respective tube opening of the support plate. The support plate supports the tubes of the tube bundle in the tube openings transversely to the longitudinal direction of the tubes.

This has the advantage that the tubes of the tube bundle can individually thermally expand, but vibrations caused by certain flow states and bending of the tubes can be avoided. A further advantage is that all the tubes of the tube bundle are supported by a support plate. A directly adjacent further support plate, which supports the remaining tubes of the tube bundle, is therefore not necessary.

The tubes of the tube bundle are preferably not axially fixed in place in the respective tube opening of the support plate. The tubes of the tube bundle are in this case axially movable in the axial direction through a respective tube opening of the support plate. Locally different temperatures can result in locally different expansions of the tubes. Since the tubes are axially movable, individual tubes are not disrupted in their individual expansion by the support plates. Movements transverse to the longitudinal axis are, however, minimized.

The shape of the support plate can be adapted to that of the reactor vessel. The support plate preferably has a rectangular shape with rounded corners. The support plate particularly preferably has an elliptical shape, in particular a circular shape.

The support plate can adversely affect the fluid exchange between the reactor-vessel part located above the support plate and the reactor-vessel part located below the support plate. In order to keep this effect as small as possible, the support plate has multiple fluid-exchange cutouts between the tube openings. The fluid exchange takes place in particular between reactor-vessel regions between which the support plate is disposed. The free flow cross section in the reactor vessel can be determined by the cutouts in the support plate. The free flow cross section is that cross section of a tube or channel through which a medium flows. The free flow cross section thus denotes an area through which a medium can flow.

The greater the overall area of the cutouts, the greater the free flow cross section. On the other hand, the free flow cross section may also be determined via the pitch. The pitch describes the distance from tube to tube. The greater the distance between tubes, the greater the free flow cross section without a support plate. The pitch can thus define a maximum area of the cutouts.

Given a fixed number of cutouts, the free flow cross section can be increased by making the individual cutouts bigger.

The reactor vessel may have multiple support plates. Multiple support plates are preferably distributed along the longitudinal axis in the reactor vessel. The support plates are preferably uniformly distributed along the longitudinal axis in the reactor vessel. The support plates are preferably each designed like the support plate described herein. However, it is also possible for the device to have, in addition to a support plate designed as described herein, one or more further support plates or comparable elements which are not designed like the support plate designed as described herein.

The device has the advantage that the hindering effect of the support plate is minimized and at the same time the tubes are prevented from bending, bowing or swaying during transport or during operation. The hindering effect of the support plate can be minimized by improving the free flow cross section through the cutouts.

In a preferred embodiment of the device, the support plate, for at least some of the cutouts of the first support plate, forms a respective divider in each case between the cutout and the closest tube opening to the cutout. The dividers have a respective minimum width, which is the same for at least some of the dividers, preferably for all the dividers.

The support plate should on the one hand be stable enough to sufficiently support the tubes. This can be achieved by the dividers having a sufficient width. On the other hand, the support plate should adversely affect the fluid exchange as little as possible. This can be achieved by the dividers being kept as small as possible. Assessing the two aforementioned conditions will make it possible to determine how wide a divider should optimally be at its narrowest point, and thus how large the minimum width of a divider should optimally be. In the present embodiment, at least some of the dividers, preferably even all the dividers, have the same minimum width. In this way, the best possible compromise between stability and fluid exchange can be achieved for all these dividers.

In a further, preferred embodiment of the device, at least some of the cutouts each lie centrally between three tube openings that are adjacent to one another in pairs.

The cutouts may for example each lie centrally between three adjacent tube openings, the centres of these three tube openings preferably forming an equilateral triangle.

At least some of the cutouts are preferably passage bores. The advantage of passage bores is that the support plate can be produced particularly easily and has a very high strength and stiffness.

In a further, preferred embodiment of the device, the support plate has intermediate regions, which are each formed between three tube openings that are adjacent to one another in pairs, and wherein at least some of the cutouts of the support plate are restricted to a respective one of the intermediate regions.

The advantage of this embodiment is that the area of the cutout can be increased given an invariable pitch. Figuratively speaking, the intermediate region between the tube openings can be utilized better. This makes it possible to further reduce the hindering effect of the support plates.

In a further, preferred embodiment of the device, at least some of the intermediate regions are star-shaped.

A star-shaped intermediate region is to be understood in the mathematical sense in this case. An intermediate region is star-shaped when there is a point, what is referred to as the centre of the star, from which all other points of the intermediate region are visible. That means that every line connecting a point to the centre of the star lies completely in the intermediate region. The centre of the star can, figuratively speaking, “see” all sides of the cutout.

At least some of the intermediate regions may preferably have the shape of triangular holes. A triangular hole in this case means a three-sided hole with rounded corners and concave sides. A triangular hole is star-shaped in the sense of the term star-shaped that is used herein.

The advantage of this embodiment is that the area of the cutout can be increased given an invariable pitch. Figuratively speaking, the available space between the tube openings can be utilized better. This makes it possible to further reduce the hindering effect of the support plate.

In a further, preferred embodiment of the device, the support plate has intermediate regions, which are each formed between three tube openings that are adjacent to one another in pairs, and wherein at least some of the cutouts of the support plate each extend over at least two intermediate regions that are adjacent to one another.

This configuration can be obtained by removing the respective connecting divider between two adjacent intermediate regions. In this configuration, for example three cutouts may be formed around a tube opening. For each cutout, two respective adjacent intermediate regions may be connected. Each cutout can have four adjacent tube openings. The four tube openings may as a group have a diamond shape.

The advantage of this embodiment is that the hindering effect of the support plate can be reduced. The free flow cross section is relatively high in spite of a small pitch of the tubes. Furthermore, the support plate can be made more lightweight by this embodiment.

In a further, preferred embodiment of the device, the support plate encompasses all the tubes of a tube bundle.

The advantage of this embodiment is that the support plate supports all the tubes. This prevents individual tubes from swaying or bowing during operation or during transport.

A further aspect of the invention sets out an assembly for methanol synthesis. The assembly comprises the described device.

The described advantages and features of the device can be applied and transferred to the assembly.

To produce methanol, a synthesis gas can be converted by reaction over catalysts to yield methanol. The synthesist gas can contain reaction reactants for the methanol synthesis.

Besides the described device, the assembly preferably comprises a source for reaction reactants for the methanol synthesis. The assembly may also comprise multiple devices designed as described and arranged for example in series.

A further aspect of the invention sets out a process. In the process, reaction reactants for the methanol synthesis are conducted through the tubes of the tube bundle. A cooling medium is conducted through the reactor vessel outside the tubes of the tube bundle. The cooling medium is preferably liquid water and/or steam.

In an alternative embodiment of the process, the reaction reactants for the methanol synthesis are conducted through the reactor vessel around the tubes of the tube bundle. A cooling medium is then conducted through the tubes of the tube bundle. The cooling medium is preferably gaseous.

The described advantages and features of the device can be applied and transferred to the process, and vice versa. The device is preferably designed for operation according to the described process. The process is preferably performed with the device.

A further aspect of the invention sets out the use. The described device is used for methanol synthesis. Reaction reactants are converted to methanol in the device.

The described advantages and features of the device, the assembly and the method can be applied and transferred to the use, and vice versa.

The device is preferably used for water-cooled methanol synthesis. As an alternative, it is preferred to use the device for gas-cooled methanol synthesis.

In water-cooled methanol synthesis, the tubes are cooled with water, while a synthesis gas is converted to methanol in the tubes. The water discharges the heat from the tubes and partially evaporates. The rising bubbles of steam can flow through the cutouts of the support plate.

In gas-cooled methanol synthesis, the tubes are cooled by a cooler gas in the tubes. The synthesis gas is converted to methanol outside the tubes. The heat is transferred to the tubes and discharged by the gas in the tubes. The synthesis gas can flow through the cutouts of the support plate.

### BRIEF DESCRIPTION OF THE DRAWINGS

The invention is elucidated in detail hereinafter with reference to the figures. The figures show particularly preferred exemplary embodiments but the invention is not limited to these. The figures and the size ratios represented therein are merely schematic. In the figures:

FIG. 1: shows a schematic view of an assembly according to the invention for methanol synthesis,

FIG. 2: shows a schematic plan view of a first configuration of a support plate, as can be used in the assembly from FIG. 1,

FIG. 3: shows a sectional representation of the support plate from FIG. 2,

FIG. 4: shows a view of a detail of a second configuration of a support plate, as can be used in the assembly from FIG. 1,

FIG. 5: shows a view of a detail of a third configuration of a support plate, as can be used in the assembly from FIG. 1,

FIG. 6: shows a view of a detail of a fourth configuration of a support plate, as can be used in the assembly from FIG. 1.

### DETAILED DESCRIPTION OF PREFERRED EMBODIMENTS

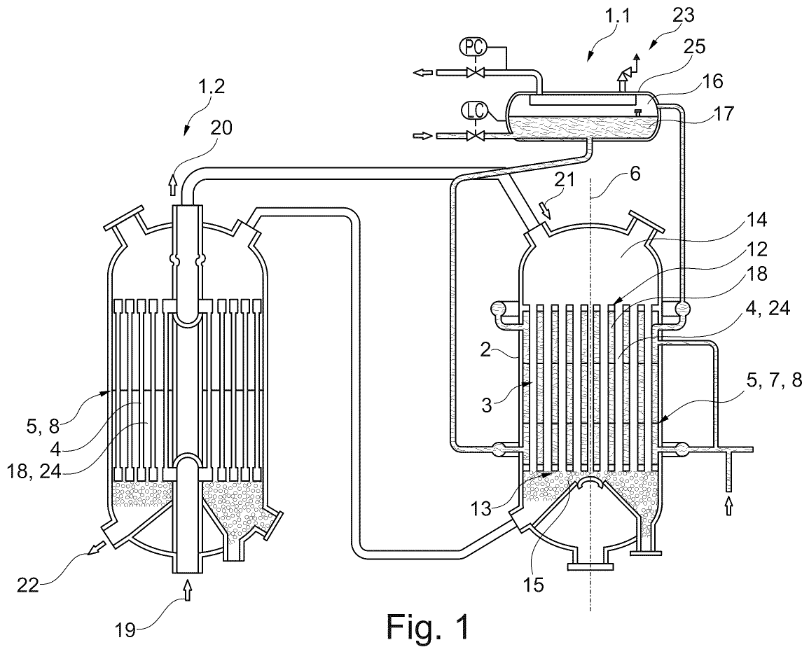

FIG. 1 shows a schematic view of an assembly 23 for methanol synthesis. Synthesis gas is conducted through two devices 1.1; 1.2 connected in series. The devices 1.1; 1.2 are used for methanol synthesis.

Pre-heated synthesis gas is fed to the first device 1.1 through a gas inlet 21. The first device 1.1 comprises a reactor vessel 2, a tube bundle 3 of multiple tubes 4, and multiple support plates 5. The tube bundle 3 is located in the reactor vessel 2. The support plates 5 are each disposed in the reactor vessel 2 transversely to a longitudinal axis 6 of the reactor vessel 2, wherein each tube 4 of the tube bundle 3 is routed through a respective tube opening 7 of the support plate 5. The support plates 5 support the tubes 4 of the tube bundle 3 in the tube openings 7 transversely to the longitudinal direction of the tubes 4. Between the tube openings 7, the support plates have fluid-exchange cutouts 8.

The tubes 4 are connected at a first end 12 and at a second end 13 to a respective tube end plate. The reactor vessel 2 and the tubes 4 of the tube bundle 3 are upright. The first end 12 is arranged at the top and the second end 13 at the bottom of the reactor vessel 2.

The synthesis gas is distributed in a distributor 14 among the tubes 4 of the tube bundle 3.

The tubes 4 contain a catalyst 24. The synthesis gas contains reaction reactants. The reaction reactants for the methanol synthesis are conducted through the tubes 4 of the tube bundle 3. The synthesis gas in the tubes 4 is partially converted to methanol. A cooling medium is conducted through the reactor vessel 2 outside the tubes 4 of the tube bundle 3. The heat released from the exothermic reaction in the tube 4 is transferred via the tube wall to the cooling medium located in a shell space 18. While the catalyst 24 in the tube 4 is cooled by this process, energy is fed to the cooling medium. The cooling medium used is water, which is fed to the shell space 18 on the second side 13. Resulting bubbles of steam and the boiling two-phase water mixture flow vertically upwards owing to differences in density. The steam flows through the cutouts 8 of the support plate 5 with small pressure drops. The steam is collected at the top and fed to a water condenser 25. The reservoir of the condenser 25 contains saturated steam 16 and water 17 at or slightly below boiling point. The water 17 is fed to the first device 1.1 on the second side 13 and distributed throughout the shell space 18 of the reactor vessel 2. The cooling thus works on the thermo-siphon effect.

Product gas and the partially unreacted synthesis gas are collected from the tube bundle 3 in a collector 15. The gas mixture then flows to the second device 1.2.

In the second device 1.2, which is downstream of the first device 1.1, synthesis gas from a synthesis gas inlet 19 is pre-heated.

The second device 1.2 has a partially similar structure to the first device 1.1.

By contrast to the first device 1.1, the second device 1.2 contains, between the tubes 4 of the second device 1.2 in the shell space 18, a catalyst 24 in the form of a catalyst bed.

The synthesis gas, which comprises the reaction reactants, is conducted through the reactor vessel 2 through the shell space 18 for the methanol synthesis outside the tubes 4 of the tube bundle 3 and a cooling medium is conducted through the tubes 4 of the tube bundle 3. The synthesis gas in the shell space 18 flows downwards to a product gas outlet 22 with small pressure drops through the cutouts 8 of the support plate 5.

The heat released from the exothermic reaction in the shell space 18 is transferred via the tube wall to the cooling medium located in the tube 4. While the catalyst 24 in the shell space 18 is cooled by this process, energy is fed to the cooling medium. The cooling medium used is synthesis gas, which is fed to the tubes 4 via the synthesis gas inlet 19. The energy fed in causes the synthesis gas from the synthesis gas inlet 19 to be heated and it is passed in the form of pre-heated synthesis gas 20 to the gas inlet 21 of the first device 1.1.

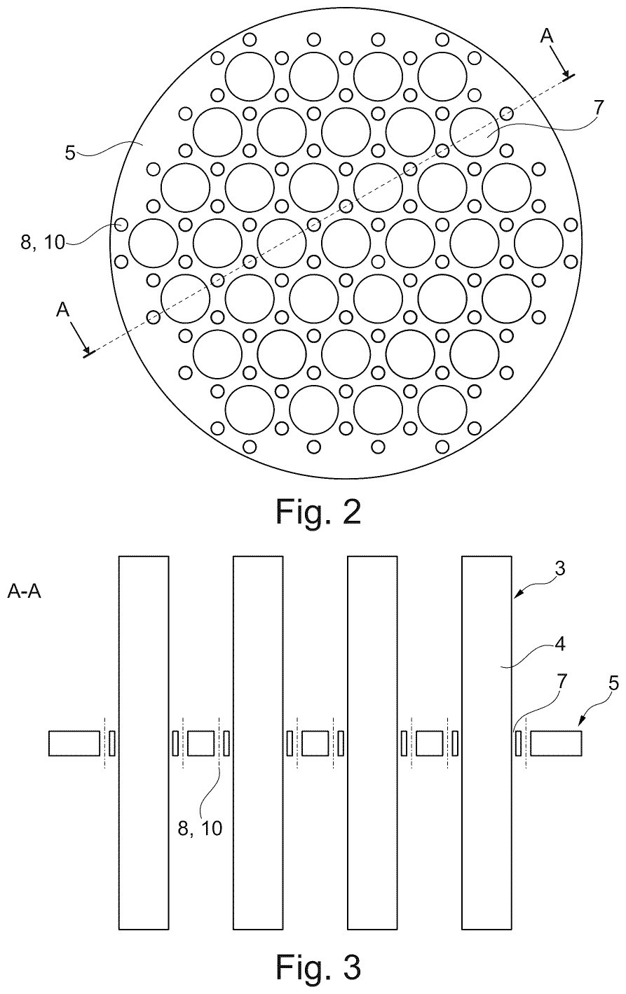

FIG. 2 shows a schematic plan view of a first configuration of a support plate 5, as can be used in the devices 1.1; 1.2 from FIG. 1. The support plate 5 has fluid-exchange cutouts 8 between the tube openings 7. The cutouts 8 are produced as passage bores 10.

FIG. 3 shows a sectional representation of the support plate 5 from FIG. 2. Each tube 4 of the tube bundle 3 is routed through a respective tube opening 7 of the support plate 5. The support plate 5 supports the tubes 4 of the tube bundle 3 in the tube openings 7 transversely to the longitudinal direction of the tubes 4.

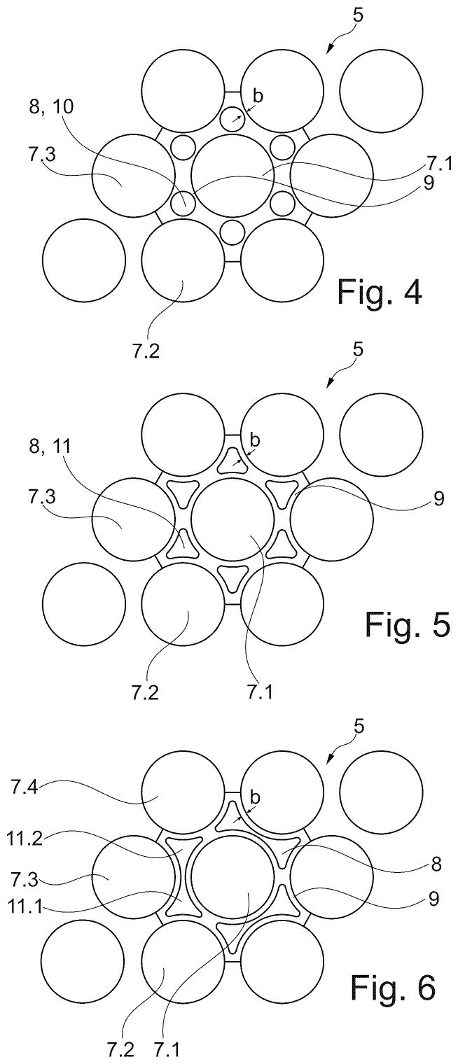

FIG. 4 shows a view of a detail of a second configuration of a support plate 5, as can be used in the devices 1.1; 1.2 from FIG. 1. The support plate 5 forms, for at least some of the cutouts 8, a respective divider 9 between the cutout 8 and the closest of the tube openings 7.1-7.3 to the cutout. The dividers 9 have a respective minimum width b, which is the same for at least some of the dividers 9. The cutouts 8 each lie centrally between three tube openings 7.1-7.3 that are adjacent to one another in pairs. The cutouts 8 are produced as passage bores 10.

FIG. 5 shows a view of a detail of a third configuration of a support plate 5, as can be used in the devices 1.1; 1.2 from FIG. 1. The support plate 5 forms, for at least some of the cutouts 8, a respective divider 9 between the cutout 8 and the closest of the tube openings 7.1-7.3 to the cutout. The dividers 9 have a respective minimum width b, which is the same for all the dividers 9. The cutouts 8 each lie centrally between three tube openings 7.1-7.3 that are adjacent to one another in pairs. The support plate 5 has intermediate regions 11, which are each formed between three tube openings 7.1-7.3 that are adjacent to one another in pairs. The cutouts 8 of the support plate 5 are restricted to a respective one of the intermediate regions 11. The intermediate regions 11 are star-shaped.

FIG. 6 shows a view of a detail of a fourth configuration of a support plate 5, as can be used in the devices 1.1; 1.2 from FIG. 1. The support plate 5 forms, for at least some of the cutouts 8, a respective divider 9 between the cutout 8 and the closest of the tube openings 7.1-7.4 to the cutout. The dividers 9 have a respective minimum width b, which is the same for all the dividers 9.

The support plate 5 has intermediate regions 11.1; 11.2, which are each formed between three tube openings 7.1-7.4 that are adjacent to one another in pairs, and wherein at least some of the cutouts 8 of the support plate 5 each extend over at least two intermediate regions 11.1; 11.2 that are adjacent to one another. The intermediate regions 11.1, 11.2 are star-shaped.

### LIST OF REFERENCE NUMERALS

1.1; 1.2 Device
2 Reactor vessel
3 Tube bundle
4 Tube
5 Support plate
6 Longitudinal axis
7; 7.1-7.4 Tube opening
8 Cutout
9 Divider
10 Passage bore
11; 11.1; 11.2 Intermediate region
12 First end
13 Second end
14 Distributor
15 Collector
16 Saturated steam
17 Water slightly below or at boiling point
18 Shell space
19 Synthesis gas inlet
20 Pre-heated synthesis gas
21 Gas inlet
22 Product gas outlet
23 Assembly
24 Catalyst
25 Condenser
b Minimum width

1. A device (1.1, 1.2) comprising a reactor vessel (2), a tube bundle (3) of multiple tubes (4), and at least one support plate (5),
wherein the tube bundle (3) is disposed in the reactor vessel (2),
wherein the support plate (5) is disposed in the reactor vessel (2) transversely to a longitudinal axis (6) of the reactor vessel (2),
wherein each tube (4) of the tube bundle (3) is routed through a respective tube opening (7) of the support plate (5),
wherein the support plate (5) supports the tubes (4) of the tube bundle (3) in the tube openings (7) transversely to the longitudinal direction of the tubes (4),
wherein the support plate (5) has a plurality of fluid-exchange cut-outs (8) between the tube openings (7).

2. The device (1.1; 1.2) according to claim 1, wherein the support plate (5), for at least some of the cut-outs (8), forms a respective divider (9) between the at least some cut-outs (8) and the closest tube opening (7) to the cut-out, and wherein the dividers (9) have a respective minimum width (b) which is the same for at least some of the dividers (9).

3. The device (1.1; 1.2) according to claim 1, wherein at least some of the cut-outs (8) each lie centrally between three tube openings (7.1-7.3) that are adjacent to one another in pairs.

4. The device (1.1; 1.2) according to claim 1, wherein the support plate (5) has intermediate regions (11), which are each formed between three tube openings (7.1-7.3) that are adjacent to one another in pairs, and wherein at least some of the cut-outs (8) of the support plate (5) are restricted to a respective one of the intermediate regions (11).

5. The device (1.1; 1.2) according to claim 4, wherein at least some of the intermediate regions (11) are star-shaped.

6. The device (1) according to claim 4, wherein the support plate (5) has intermediate regions (11.1; 11.2), which are each formed between three tube openings (7.1-7.4) that are adjacent to one another in pairs, and wherein at least some of the cut-outs (8) of the support plate (5) each extend over at least two intermediate regions (11.1; 11.2) that are adjacent to one another.

7. The device (1.1; 1.2) according to claim 1, wherein the support plate (5) encompasses all the tubes (4) of the tube bundle (3).

8. A process for methanol synthesis using the device (1.1; 1.2) according to claim 1, wherein reaction reactants for the methanol synthesis are conducted through the tubes (4) of the tube bundle (3) and a cooling medium is conducted through the reactor vessel (2) outside the tubes (4) of the tube bundle (3),
or
wherein reaction reactants for the methanol synthesis are conducted through the reactor vessel (2) outside the tubes (4) of the tube bundle (3) and a cooling medium is conducted through the tubes (4) of the tube bundle (3).
